(19) United States
(12) Patent Application Publication
(10) Pub. No.: US 2025/0040791 A1
(43) Pub. Date: Feb. 6, 2025

(54) MEDICAL ASSEMBLIES, DEVICES, SYSTEMS, AND RELATED METHODS FOR WETTING A MEDICAL INSTRUMENT

(71) Applicant: Boston Scientific Scimed, Inc., Maple Grove, MN (US)

(72) Inventors: Kurt Nicholas ROBAKIEWICZ, Upton, MA (US); Christopher A. BENNING, Hopkinton, MA (US); Ryan V. WALES, Northborough, MA (US); Scott E. BRECHBIEL, Burlington, MA (US); John B. GOLDEN, Norton, MA (US); Paul SMITH, Smithfield, RI (US)

(73) Assignee: Boston Scientific Scimed, Inc., Maple Grove, MN (US)

(21) Appl. No.: 18/792,676

(22) Filed: Aug. 2, 2024

Related U.S. Application Data
(60) Provisional application No. 63/517,640, filed on Aug. 4, 2023.

(51) Int. Cl. A61B 1/00; A61B 1/005; A61B 1/018

(52) U.S. Cl. CPC A61B 1/00128; A61B 1/00068; A61B 1/00131; A61B 1/0052; A61B 1/018

(57) ABSTRACT

A medical system may include a fluid source, a fluid reservoir, and a valve. The valve may be coupled to the fluid source and the fluid reservoir so as to permit fluid flow between the fluid source and the fluid reservoir. The valve may include a first opening configured to receive a medical instrument. Upon insertion of the medical instrument into the first opening of the valve, the medical instrument may be lubricated with a fluid from the fluid source.

### CROSS-REFERENCE TO RELATED APPLICATIONS

This application claims the benefit of priority under 35 U.S.C. § 119 from U.S. Provisional Application No. 63/517,640, filed Aug. 4, 2023, which is incorporated by reference herein in its entirety.

### TECHNICAL FIELD

This disclosure generally relates to medical devices, systems, and related methods for wetting a medical instrument. More particularly, at least some embodiments of the present disclosure relate to devices, systems, and related methods for lubricating a medical instrument prior to insertion into a body lumen of a subject.

### BACKGROUND

Medical instruments/devices are often inserted into a subjects body to perform a therapeutic and/or diagnostic procedure inside the subjects body. An example of such a device is a catheter, which is introduced into the body for accessing various parts of the subjects anatomy and/or delivering therapy to a target tissue. A catheter is inserted into the subjects body through an opening (e.g., a natural opening or an incision) and is delivered to the target tissue site inside the body, for example, through a body lumen. In one example, a catheter may be inserted directly into a subjects mouth and advanced through the subjects esophagus, to a target site. In another example, a catheter may be inserted into the subject via a secondary medical device such as, for example, a device having a working channel (e.g., a scope such as an endoscope).

Prior to inserting the catheter into the subject, one or more portions of the catheter or other medical instrument/device may be wetted, or lubricated. Wetting one or more portions of the catheter or other medical device/instrument prior to inserting the catheter into the subject may assist in enhancing the subjects comfort during a procedure. For example, lubricating the catheter may assist in reducing friction between the catheter or other medical device/instrument and the subject and/or between the catheter and the secondary medical device. Therefore, medical systems, devices, and related methods are needed for wetting or lubricating a medical instrument during a medical procedure.

### SUMMARY

Examples of this disclosure relate to, among other things, systems, devices, and methods for performing one or more medical procedures with medical devices. Specifically, this disclosure includes medical systems, devices, and methods for wetting a medical instrument. Each of the examples disclosed herein may include one or more of the features described in connection with any of the other disclosed examples.

According to one aspect, a medical system may include a fluid source, a fluid reservoir, and a valve. The valve may be coupled to the fluid source and the fluid reservoir so as to permit fluid flow between the fluid source and the fluid reservoir. The valve may include a first opening configured to receive a medical instrument. Upon insertion of the medical instrument into the first opening of the valve, the medical instrument may be lubricated with a fluid from the fluid source.

In some aspects, the medical system may further include an adapter. A first end of the adapter may be coupled to the first opening of the valve. A second end of the adapter may include an opening configured to receive the medical instrument. The adapter may include a gate configured to prevent fluid flow from the first end of the adapter to the second end of the adapter. The gate may include a first end and a second end. The first end may be fixed to the wall of the adapter. The second end of the gate may be free.

In some aspects, in a first configuration, the second end of the gate may abut a wall of a lumen of the adapter. Upon insertion of the medical instrument into the adapter and through the gate, the gate may transition to a second configuration. In the second configuration, the second end of the gate may abut a surface of the medical instrument. In the second configuration, the gate may be configured to prevent fluid flow from the first end of the adapter to the second end of the adapter. In some aspects, the second end of the gate may be between the first end of the adapter and the first end of the gate.

In some aspects, the adapter may include a channel extending through a wall of the adapter. The channel may extend along at least a portion of a length of the adapter. In some aspects, the adapter may be integrally formed with the valve.

In some aspects, the valve may further include a first arm, a second arm, a third arm, and a fourth arm. The first arm may be coupled to the fluid source. The second arm may be coupled to the fluid reservoir. The third arm may include the first opening configured to receive the medical instrument. The fourth army may include a second opening. Each of the first arm, the second arm, the third arm, and the fourth arm may be fluidly connected.

In some aspects, a longitudinal axis of the third arm may be angled in a first direction relative to a longitudinal axis of the first arm. The longitudinal axis of the fourth arm may be angled in a second direction, relative to the longitudinal axis of the first arm. The longitudinal axis of the first arm and a longitudinal axis of the fourth arm may be parallel or coaxial. The longitudinal axis of the second arm and the longitudinal axis of the third arm may be parallel or coaxial.

In some aspects, the first direction of the first arm may be opposite the second direction of the fourth arm.

In some aspects, the fluid reservoir may be fluidly coupled to a medical device.

In some aspects, a conduit may be coupled to the second opening of the fourth arm. The conduit may include a first branch, a second branch, and a third branch. The first branch may be coupled to the fourth arm. The second branch may include an opening configured to receive a second medical instrument. The third branch may be coupled to an opening of a medical device having a working channel.

According to other aspects, a medical system may include a fluid container and a dipping container. The dipping container may be configured to receive at least a portion of a medical instrument. The dipping container may include a first opening in a bottom or a side of the dipping container. The dipping container may include a second opening configured for receiving a medical instrument. The fluid container may be fluidly coupled to the dipping container via the first opening. In some aspects, the dipping container may be movable relative to the fluid container.

In some aspects, the medical system may further include a stopper. The stopper may be configured to prevent fluid flow to the dipping container in a first configuration. The stopper may be configured to permit fluid flow to the dipping container in a second configuration.

In some aspects, the medical system may further include a coupler fixed to the dipping container. The coupler may be configured to removably couple the dipping container to the fluid container.

In some aspects, a wall of the fluid container may define a wall of the dipping container.

According to other aspects, a medical device may include a bridge, a base, an arm, and a gap between the base and the arm. The base may extend laterally from the bridge in a first direction. The arm may extend laterally from the bridge in the first direction. The arm may include a nozzle configured to deliver a fluid into the gap. Upon insertion of a medical instrument through the gap, the fluid may applied to a surface of the medical instrument.

In some aspects, the medical device includes a controller, wherein the controller is configured to control delivery of the fluid.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a perspective view of a medical system according to aspects of this disclosure.

FIG. 2 illustrates a front view of an exemplary system for wetting a medical instrument, according to aspects of this disclosure.

FIG. 3A illustrates a front view of a portion of the exemplary system shown in FIG. 2, according to aspects of this disclosure.

FIGS. 3B and 3C illustrate a cross-section of the portion of the exemplary system shown in FIG. 3A in a first configuration (FIG. 3B) and in a second configuration (FIG. 3C), according to aspects of this disclosure.

FIG. 4 illustrates a front view of an alternative exemplary system for wetting a medical instrument, according to aspects of this disclosure.

FIG. 5 illustrates a front view of a further alternative exemplary system for wetting a medical instrument, according to aspects of this disclosure.

FIG. 6 illustrates a front view of an alternative exemplary system for wetting a medical instrument, according to aspects of this disclosure.

FIGS. 7A-7C illustrate a portion of an alternative exemplary system for wetting a medical instrument in a first configuration (FIG. 7A), a second configuration (FIG. 7B), and a third configuration (FIG. 7C), according to aspects of this disclosure.

FIGS. 8A and 8B illustrate a front view (FIG. 8A) and a side view (FIG. 8B) of a further alternative system for wetting a medical instrument, according to aspects of this disclosure.

FIG. 9 illustrates a front view of an exemplary device for wetting a medical instrument, according to aspects of this disclosure.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when placing the device into the subject. Proximal and distal directions are labeled with arrows marked “P” and “D,” respectively, throughout various figures.

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic. Additionally, terms that indicate the geometric shape of a component/surface encompass both exact and approximate shapes.

Medical devices/instruments, such as, for example, scopes, catheters, guidewires, baskets, forceps, or any other medical device are often inserted into a subjects body to perform a therapeutic and/or diagnostic procedure inside a subjects body. Herein, the phrases “medical device” and “medical instrument” are used interchangeably. The medical devices may be inserted directly into the subjects body (e.g., via an orifice in a subject) and/or inserted through another medical device, for example, a medical device having a working channel, such as an endoscope or other type of scope. Insertion of the medical devices into the subject and/or into the other medical device may be difficult due to frictional forces between the medical devices and/or between the medical device and the subjects tissue. These frictional forces may result in discomfort for the subject, injury to the subject, extended procedural time, among other undesirable results. To overcome these frictional forces between the medical devices and/or between the medical device and the subject, a surface of the medical device is often lubricated.

Currently, to wet or lubricate a medical device, a portion of the medical device (e.g., the distal portion) is immersed into an open container of water, saline, or other lubricant. The container may be placed on a tower or a flat surface (e.g., a counter, a table, etc.), for example, in a procedure room. The container may occupy valuable space in the procedure room, and having an open container in the procedure room increases the likelihood the water or lubricant will spill onto the floor or another surface. Spillage may be hazardous to the electrical equipment and/or to occupants of the procedure room.

This disclosure is drawn to medical systems, devices, and related methods for wetting, or lubricating, a surface of a medical device/instrument. Wetting one or more portions of the medical device prior to inserting the medical device into the subject or into the working channel of another medical device may assist in enhancing subject comfortability during a procedure, among other benefits. For example, lubricating the device may assist in reducing friction between the device and the subject and/or between the device and a secondary medical device. Some of the embodiments described herein utilize a fluid source used for other aspects of a medical procedure (e.g., a source of irrigation/lens wash) as a source of fluid for lubricating the medical device. The aspects herein may provide for a source of lubricating fluid without requiring an open container.

This disclosure is described with reference to exemplary medical systems for lubricating, or wetting, a medical instrument. This may provide improved medical device/instrument functionality and/or assist medical professionals with performing medical procedures. However, it should be noted that reference to any particular device and/or any particular procedure is provided only for convenience and not intended to limit the disclosure. A person of ordinary skill in the art would recognize that the concepts underlying the disclosed systems, devices and application methods may be utilized in any suitable procedure, medical or otherwise. The devices, assemblies and systems described herein may be used in conjunction with other types of medical devices. This disclosure may be understood with reference to the following description and the appended drawings, wherein like elements are referred to with the same reference numerals.

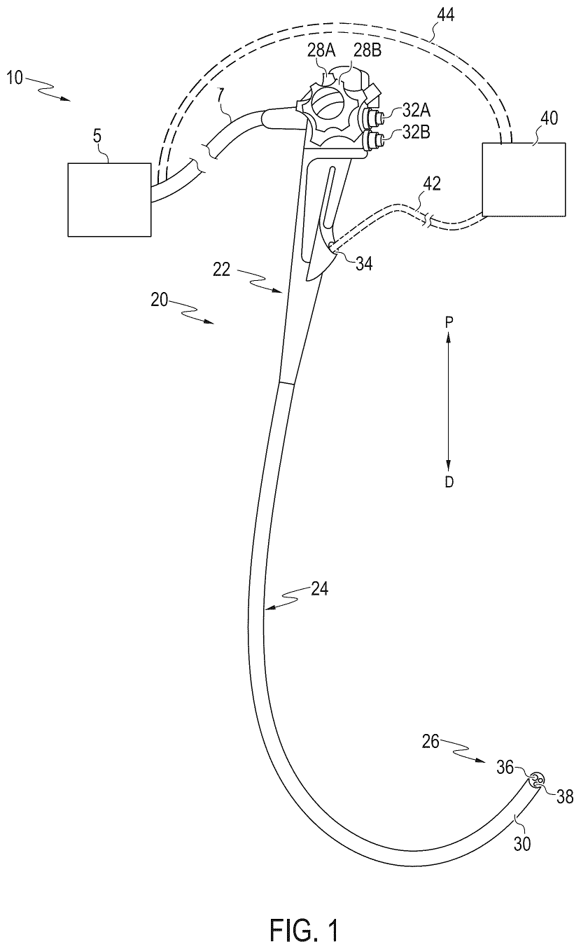

Referring to FIG. 1, a medical system 10 according to an exemplary embodiment is shown. Medical system 10 may include a medical device 20, a wetting system 40, and a processing unit 5. Medical device 20 may include a handle 22 and an insertion portion 24 (e.g., a shaft or a catheter). Insertion portion 24 may be connected to a distal portion of handle 22. Insertion portion 24 may terminate distally in a distal tip 26. An umbilicus 7 may extend from a proximal portion of handle 22 (or another portion of handle 22).

Umbilicus 7 may be removably coupled (e.g., directly or indirectly) to processing unit 5. Processing unit 5 may be configured to process information (e.g., sensor data, imaging data, light data, etc.) received from medical device 20. In some aspects, processing unit 5 may be a controller associated with medical device 20. Umbilicus 7 may include one or more electrical cables and/or light cables for coupling to processing unit 5 via, e.g., a removable connector. Although not shown, processing unit 5 may include a visual output (e.g., an internal monitor or screen) or processing unit 5 may be coupled to a visual output (e.g., an external or separate monitor or screen). Although not shown, umbilicus 7 may additionally or alternatively include one or more lumens for supplying gas or liquid to handle 22 and/or insertion portion 24.

Handle 22 may include a first actuator 28A and/or a second actuator 28B. First actuator 28A and/or second actuator 28B may include, for example, rotatable knobs that rotate to push/pull one or more elements that extend through insertion portion 24 and connect to a distal end portion 30 of insertion portion 24. For example, first actuator 28A and/or second actuator 28B may be configured to rotate about their axes to push/pull actuating elements (e.g., wires, not shown) which extend within one or more lumens of insertion portion 24. Rotation of first actuator 28A and/or second actuator 28B may cause insertion portion 24 and/or distal end portion 30 to bend, for example, via an articulating joint (not shown). Additionally or alternatively, handle 22 may include additional actuators (e.g., buttons, knobs, levers, locks, etc.) to, for example, limit movement of the first actuator 28A and/or second actuator 28B, close or open a forceps, rotate an end effector about a longitudinal axis, raise or lower an elevator, capture an image, and/or provide other functionality to an end effector and/or distal end portion 30.

Handle 22 may also include a first valve 32A and a second valve 32B. Although two valves (i.e., first valve 32A and second valve 32B) are shown, handle 22 may include additional (e.g., a third valve, a fourth valve, etc.) or fewer valves (e.g., no valves or only first valve 32A). In some embodiments, first valve 32A may be configured to control the supply of air and/or water to distal end portion 30. Second valve 32B may be configured to control the application of suction to distal end portion 30. Additional valves may be used, for example, to control the application of one or more medicines, agents, materials, etc. to distal end portion 30.

Handle 22 may also include a proximal port/opening 34, which may be fluidly connected to one or more lumens (e.g., working channel(s)) of insertion portion 24. For example, a medical instrument (not shown in FIG. 1) may be inserted into proximal opening 34 and may be extended to distal end portion 30 via the one or more lumens. The lumen(s) may have one or more distal openings 36 at distal tip 26, which may be a distalmost end of distal end portion 30. Distal opening(s) 36 may be fluidly coupled to proximal opening 34. In such a way, a medical instrument inserted into proximal opening 34 may be extended distally through distal opening 36 and extend distally from distal opening 36. Additionally or alternatively, one or more materials (e.g., liquids, gels, gasses, patches, powders, etc.) may be supplied to a target site via distal opening 36. Additionally or alternatively, suction may be supplied via proximal opening 34, for example, to remove debris from the target site via distal opening 36.

Although not shown, distal end portion 30 may include one or more articulation joints. Distal tip 26 may include one or more end effectors, and/or openings for suction, irrigation, insufflation, accessory devices, etc. Distal end portion 30 may further include one or more devices 38. Device(s) 38 may include one or more visualization devices (e.g., cameras, image sensors, lenses, etc.), one or more illumination devices (e.g., LEDs, optical fibers, etc.), one or more treatment devices (e.g., laser fibers, elevators, etc.), and/or one or more other devices to otherwise image, view, or otherwise treat a treatment site.

Wetting system 40 may be configured to wet, or lubricate, a medical instrument (not shown in FIG. 1) prior to the medical instrument being inserted into medical device 20, for example, into proximal opening 34 of handle 22 of medical device 20. For example, a medical instrument may be inserted into and/or through wetting system 40 and lubricated by wetting system 40. Once the medical instrument is lubricated by wetting system 40, the medical instrument may be inserted into proximal opening 34 and extended to distal end portion 30 of medical device 20 via one or more lumens.

In some configurations, wetting system 40 may be fluidly coupled to one or more aspects of medical device 20. For example, wetting system 40 may be fluidly coupled to proximal opening 34 via an optional conduit 42 extending between wetting system 40 and medical device 20. For example, a first end of conduit 42 may be fixedly or removably coupled to wetting system 40. A second end of conduit 42 may be fixedly or removably coupled to proximal opening 34 and/or the one or more lumens of medical device 20. In such a way, the medical instrument may be inserted into proximal opening 34 of handle 22 of medical device 20 via wetting system 40 and conduit 42. For example, the medical instrument may first be inserted into wetting system 40, where the medical instrument is lubricated. The lubricated medical instrument may then be extended through conduit 42, and then into proximal opening 34 of medical device 20.

Additionally or alternatively, in some configurations, wetting system 40 may be fluidly coupled to umbilicus 7 of medical device 20 and/or to processing unit 5. For example, conduit 42 and/or another conduit 44 (shown in dashed lines) may fluidly couple wetting system 40 to one or more portions of umbilicus 7 and/or to processing unit 5. Wetting system 40—may be used in conjunction with medical device 20 or as a standalone system. For example, the wetting system 40 and the embodiments discussed herein may be used in conjunction with any medical system or device where a medical device is to be wetted or lubricated. In some configurations, wetting system 40 may be used with an external fluid source. For example, the external fluid source may be a fluid source for irrigation/lens wash used for or in conjunction with medical device 20.

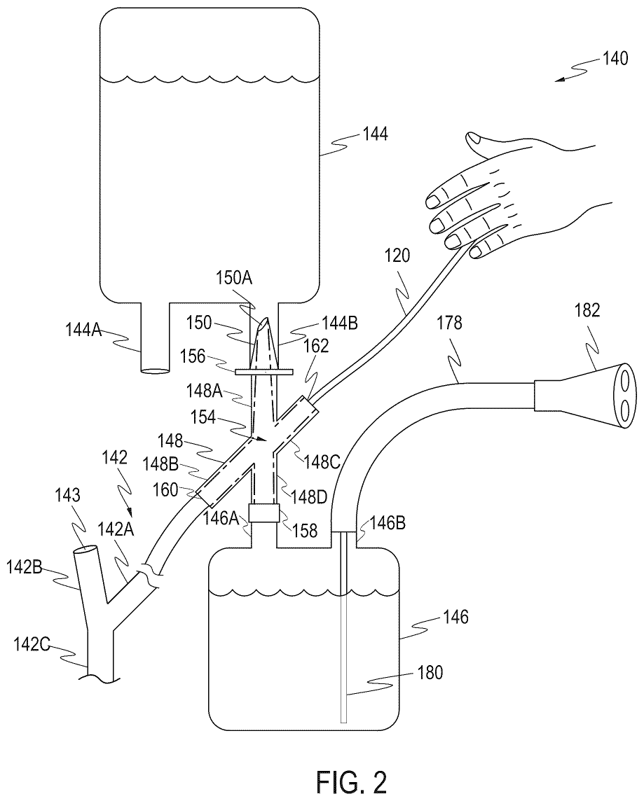

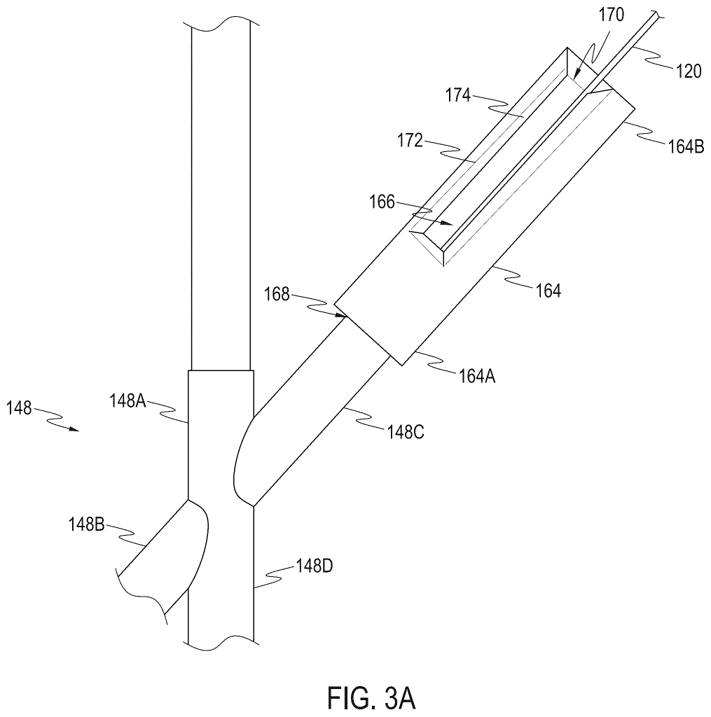

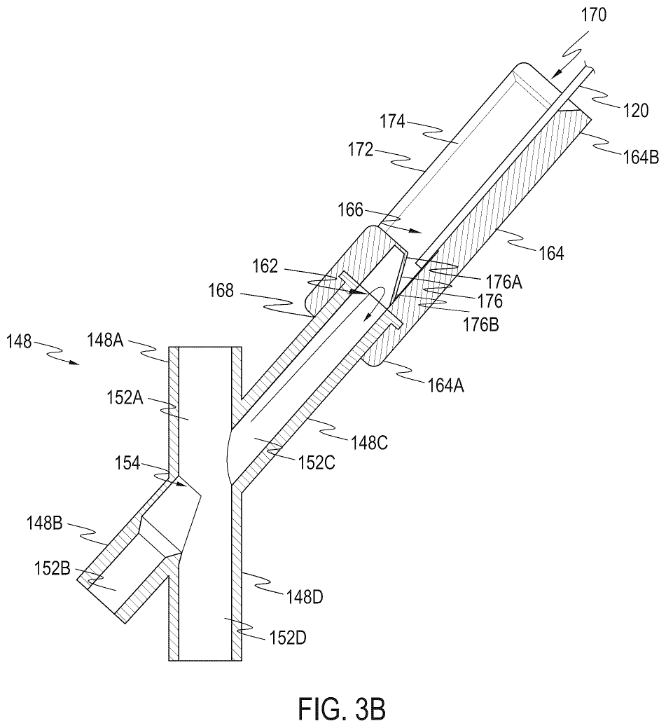

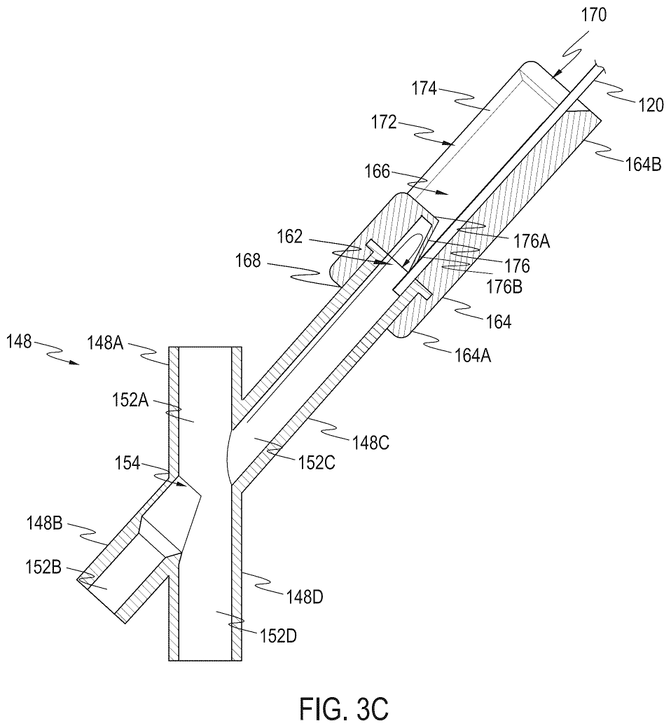

FIG. 2 illustrates a front view of an exemplary configuration of a wetting system 140 during use. Wetting system 140 includes a fluid container 144, a reservoir 146, and a valve 148. Valve 148 may be configured to allow fluid communication between fluid container 144 and reservoir 146. For example, a fluid from fluid container 144 may flow through valve 148 and into reservoir 146. FIGS. 3A-3C depict further details of valve 148.

Fluid container 144 may be any flexible or rigid container configured to contain a fluid. For example, fluid container 144 may be a water, saline, or intravenous (IV), bag or bottle. Fluid container 144 may contain a fluid (e.g., water, saline, or another fluid). Fluid container 144 may include one or more ports. For example, fluid container 144 may include a first port 144A and/or a second port 144B. One or both of first port 144A and second port 144B may be comprised of a self-sealing rubber or silicone material. For example, first port 144A and/or second port 144B may be punctured one or more times, for example, to permit fluid flow into or out of fluid container 144.

In some examples, first port 144A may be configured to permit a user to inject additional fluid into fluid container 144 and/or withdraw fluid from fluid container 144. A user may inject fluid into or withdraw fluid from fluid container 144 using, for example, a needle or an alternative injection device. In some examples, a user may inject an agent such as a medicine or another fluid into fluid container 144. The agent may be any agent in liquid form, such as, for example, a pain medicine, a coagulant, an anti-coagulant, or any other agent commonly used in endoscopic procedures. An agent may combine with another fluid already in fluid container 144 or may be the sole fluid in fluid container 144.

Second port 144B may be configured to permit fluid flow out of fluid container 144. For example, second port 144B may be configured for puncture by a spike 150 of valve 148. Spike 150 may be configured to facilitate a fluid connection between fluid container 144 and valve 148. Spike 150 may be conical having a sharp tip for puncturing second port 144B of fluid container 144. In some examples, spike 150 may be shaped similar to a needle. Spike 150 may have an opening 150A at its tip that may provide access to one or more lumens of valve 148.

Valve 148 includes a first arm 148A, a second arm 148B, a third arm 148C, and a fourth arm 148D. First arm 148A, second arm 148B, third arm 148C, and fourth arm 148D may be configured may be oriented similar to an “X” or a cross. In some examples, one of the arms (e.g., second arm 148B) may be omitted such that valve 148 has a “Y” shape. As shown in FIGS. 3B and 3C, each of first arm 148A, second arm 148B, third arm 148C, and fourth arm 148D may have a respective lumen extending longitudinally therethrough. For example, first arm 148A has a first lumen 152A, second arm 148B has a second lumen 152B, third arm 148C has a third lumen 152C, and fourth arm 148D has a fourth lumen 152D. The lumens 152A, 152B, 152C, 152D of each respective arm 148A, 148B, 148C, 148D may be in fluid communication with one another. For example, the lumens 152A, 152B, 152C, 152D may intersect at a junction 154 of valve 148. For example, a central longitudinal axis of first arm 148A may be approximately parallel to or coaxial with a central longitudinal axis of fourth arm 148D. A central longitudinal axis of second arm 148B may be approximately parallel to or coaxial with third arm 148C.

The central longitudinal axis of First arm 148A may generally extend in an upward direction. A proximal end of first arm 148A may include spike 150. First arm 148A may further include a flange 156. Flange 156 may extend radially outward, distally of spike 150. Flange 156 may be configured, for example, to prevent over-insertion of spike 150 into fluid container 144.

Fourth arm 148D may generally extend in a downward direction. In examples, fourth arm 148D may be fluidly coupled to reservoir 146 via a first connector 158. First connector 158 may be any connector commonly used in the art to fluidly connect two components. In examples, first connector 158 may contact a surface of fourth arm 148D to couple fourth arm 148D to reservoir 146. First connector may be coupled to a first port 146A of reservoir 146.

Reservoir 146 may be any flexible or rigid container configured to contain a fluid. For example, reservoir 146 may be a bag or a bottle. Fluid may flow from fluid container 144, through lumens 152A and 152D of respective arms 148A and 148D, and into reservoir 146.

Second arm 148B may extend in a first lateral direction, away from first arm 148A. Third arm 148C may extend in a second lateral direction, away from first arm 148A. The second lateral direction may be opposite of the first lateral direction. In such a way, second arm 148B and third arm 148C may be angled relative to first arm 148A. As shown in FIG. 2, an angle between second arm 148B and third arm 148C may be transverse to first arm 148A but not perpendicular to first arm 148A. Alternatively, second arm 148B and third arm 148C may each be perpendicular to first arm 148A. Second arm 148B and third arm 148C may have the same or different angles with respect to first arm 148A. In some examples, an opening 160 of second arm 148B may be oriented such that opening 160 of second arm 148B faces in a distal direction. An opening 162 (labeled in FIGS. 3B-3C) of third arm 148C may be oriented such that opening 162 of third arm 148C faces in a proximal direction. In some examples, opening 160 of second arm 148B may face in an opposite direction relative to opening 162 of third arm 148C.

As shown in FIGS. 3A-3C 148, a valve adapter 164 may be coupled to third arm 148C of valve 148. Although valve adapter 164 is not depicted in FIG. 2, it will be appreciated that the system 140 of FIG. 2 also may include valve adapter 164. FIG. 3A is a front view of valve 148 and valve adapter 164. FIGS. 3B and 3C are cross-sectional views of valve 148 and valve adapter 164 in a first configuration (FIG. 3B) and in a second configuration (FIG. 3C). Valve adapter 164 may be fixedly or removably coupled to third arm 148C of valve 148. In some examples, valve adapter 164 may be comprised of one or more flexible, stretchable, and/or semi-rigid materials so as to permit attachment to valve 148. For example, valve adapter 164 may be coupled to valve 148 via a friction fit, a snap fit, a press fit, etc. Additionally or alternatively, valve adapter 164 may be coupled to valve 148 via one or more adhesives and/or mechanical fasteners (e.g., screws, rivets, set screws, etc.). Valve adapter 164 may include a combination of materials (e.g., some rigid and some flexible) or may be made entirely of rigid materials in some aspects. In other examples, valve adapter 164 may be integrally formed with valve 148.

As shown in FIGS. 3B and 3C, valve adapter 164 may be coupled to third arm 148C such that a lumen 166 of valve adapter 164 is in fluid communication with lumen 152C extending through third arm 148C of valve 148. For example, third arm 148C may be inserted into lumen 166 of valve adapter 164, and lumen 166 of valve adapter 164 may have walls shaped to receive and mate with third arm 148C. Lumen 166 may be open on longitudinal ends thereof. Third arm 148C may be inserted into a first opening 168 on a first end 164A of valve adapter 164. A second opening 170 may be on a second end 164B (opposite first end 164A) of valve adapter 164.

A channel 172 may extend through a wall 174 of valve adapter 164, to lumen 166, such that a side of lumen 166 is open. Thus, channel 172 may provide a side opening to lumen 166 of valve adapter 164. Channel 172 may extend longitudinally along valve adapter 164, along a partial length of valve adapter 164. For example, channel 172 may extend from second opening 170 on second end 164B but may not extend to first opening 168 on first end 164A. Channel 172 may facilitate insertion of instrument 120 (discussed in further detail below) into lumen 166 and, thus, lumen 152C of third arm 148C. For example, a medical instrument 120 may be inserted into lumen 166 of valve adapter 164 via channel 172 and/or via second opening 170.

Referring to FIGS. 3B and 3C, valve adapter 164 may include a gate 176. When third arm 148C is inserted into lumen 166, gate 176 may be between opening 162 of third arm 148C and second opening 170 of valve adapter 164. In a first configuration (without medical instrument 120 inserted past gate 176), as shown in FIG. 3B, gate 176 may be configured to prevent fluid flow in a direction from first opening 162 of third arm 148C to second opening 170 of adapter 164. For example, as indicated by the curved arrow in lumen 152C of third arm 148C of FIG. 3B, fluid flow may be stopped and/or reversed by gate 176.

Gate 176 may be comprised of a flexible material. For example, gate 176 may be a rubber, a silicone, and/or a flexible plastic configured to seal or prevent fluid flow within valve adapter 164. In some examples, gate 176 may extend an entire width of lumen 166 of valve adapter 164 in the first configuration. As shown in FIGS. 3B and 3C, gate 176 may have a first end 176A that is coupled to a wall of valve adapter 164. Gate 176 may have a second, free end 176B (e.g., not coupled to a wall of adapter 164 or to another element). In the first configuration, free end 176B may touch, or abuts, the wall of adapter 164. The wall that free end 176B touches or abuts, may be, for example, opposite of the wall in which first end 176A is fixed to. First end 176A may be closer to second opening 170 than second end 176B is. Second end 176B may extend across a diameter/width of lumen 166, and away from second opening 170 and toward first opening 162 of lumen 166. In alternative aspects, gate 176 may be a septum, a one-way fluidic valve, a membrane, or another type of structure. Gate 176 may be, for example, an auto-sealing valve.

In the second configuration shown in FIG. 3C, medical instrument 120 may be inserted into lumen 166 of valve adapter 164 via second opening 170 and/or channel 172. As medical instrument 120 is inserted into lumen 166 of valve adapter 164, gate 176 may bend or distort to accommodate passage of medical instrument 120. Gate 176 may open only so far as to allow passage of medical instrument 120 and may conform to medical instrument 120. Second end 176B may be slightly spaced from an inner surface of lumen 166 in order to accommodate medical instrument 120. In such a way, gate 176 may be configured to permit insertion of medical instrument 120 through valve adapter 164 into opening 162 of third arm 148C, while preventing fluid from flowing through opening 160 into lumen 166. In such a way, gate 176 may form a seal against medical instrument 120 in the second configuration. For example, second end 176B may be configured to touch, or abut, a surface of medical instrument 120. The curved arrow of FIG. 3C illustrates the fluid flow, for example, during or after insertion of medical instrument 120 past gate 176. As shown by the arrow, the fluid flow may be stopped and/or reversed by gate 176.

Referring back to FIG. 2, a first end of a first conduit 178 may be coupled to reservoir 146, for example, via a second port 146B. A tube 180 may extend into reservoir 146 from first conduit 178. For example, tube 180 may be inserted into reservoir 146 to facilitate fluid flow from reservoir 146 into first conduit 178. A second end of first conduit 178 may include an adapter 182. Adapter 182 may be configured to couple to a pressure source (not shown) and/or to umbilicus 7 (shown in FIG. 1). In such a way, adapter 182 may be configured to permit fluid flow from reservoir 146, through tube 180 and first conduit 178, to medical device 20. In such a way, fluid from reservoir 146 may be used with medical device 20 to provide, for example, irrigation and/or lens wash.

In some examples, a second conduit 142 may be fluidly coupled to second arm 148B via opening 160. For example, second conduit 142 may be inserted into opening 160. Alternatively, arm 148B may be inserted into second conduit 142. Second conduit 142 may include a first branch 142A, a second branch 142B, and a third branch 142C. For example, first branch 142A of second conduit 142 may be fluidly coupled to second arm 148B, as described above. First branch 142A may split into second branch 142B and third branch 142C, each of which may extend from first branch 142A. Second branch 142B of second conduit 142 may include an opening/port 143. A function of second branch 142B and port 143 will be discussed in further detail below. An end of third branch 142C may be fluidly coupled to medical device 20 of FIG. 1. For example, third branch 142C of second conduit 142 may be inserted into or otherwise coupled to opening 34 of medical device 20. For example, a port defining opening 34 may be inserted into third branch 142C.

First branch 142A, second branch 142B, and third branch 142C may be arranged so that medical instrument 120 may pass from first branch 142A into second branch 142C without being diverted into third branch 142B. Similarly, branches 142A, 142B, and 142C may be arranged so that medical instrument 120 may pass from second branch 142C into third branch 142C without being diverted into first branch 142A. For example, an angle between first branch 142A and third branch 142C may be relatively open/straight. An angle between second branch 142B and third branch 142C may similarly be relatively open/straight. Meanwhile, an angle between second branch 142B and first branch 142A may be relatively sharper (e.g., an acute angle).

During use, medical instrument 120 may be inserted into valve 148 via opening 162 of third arm 148C. In some examples, valve adapter 164 may be coupled to third arm 148C of valve 148. In such an example, medical instrument 120 may first be inserted into opening 170 and/or channel 172 of valve adapter 164 and then into valve 148 via opening 162 of third arm 148C. Medical instrument 120 may be, for example, a guidewire, a catheter, or any other medical device commonly used in the art and configured for insertion into a patient.

Medical instrument 120 may pass through lumen 152C of third arm 148C, through a junction 154 between arms 148A, 148B, 148C, and 148D, and into lumen 152B of second arm 148B of valve 148. In some examples, medical instrument 120 may be extended through opening 160 of second arm 148B and inserted into second conduit 142. In aspects, medical instrument 120 may inserted into medical device 20 via opening 34 (shown in FIG. 1), coupled to conduit 142.

As medical instrument 120 is passed through valve 148, fluid from fluid container 144 may flow into opening 150A of spike 150, through lumen 152A of first arm 148A. Fluid may flow through junction 154 and into lumen 152D of fourth arm 148D, en route to reservoir 146. As fluid flows through valve 148, and in particularly through junction 154, the fluid may flow onto medical instrument 120. In such a way, medical instrument 120 is wetted or lubricated.

In the second configuration of valve adapter 164 (FIG. 3C), gate 176 may be configured to remove excess fluid from a surface of medical instrument 120, for example, as medical instrument 120 is pulled proximally and/or removed from valve adapter 164. In such a way, gate 176 may have a squeegee effect on the surface of medical instrument 120. For example, free end 176B of gate 176 may contact medical instrument 120 such that, when medical instrument 120 is pulled proximally and/or removed from valve adapter 164, fluids from the surface of medical instrument 120 are removed, or pulled off, by gate 176. The fluids removed from the surface of medical instrument 120 may then flow through lumen 152C of third arm 148C, into lumen 152D of fourth arm 148D, and into reservoir 146.

Once medical instrument 120 is pulled proximally such that medical instrument 120 is proximal of gate 176, gate 176 may transition back to the first configuration, shown in FIG. 3B. In such a way, gate 176 may be configured to transition between the first configuration and second configuration one or more times throughout a procedure. For example, as medical instrument 120 (or multiple medical instruments 120) is/are inserted and/or removed multiple times over the course of a procedure, gate 176 may be configured to transition between the first and second configurations shown in FIGS. 3B and 3C, respectively. Thus, adapter 164 may be configured to prevent or limit spillage of fluids outside of valve 148 and valve adapter 164.

After (and while) wetting medical instrument 120 with fluid from fluid container 144, excess fluid may flow from valve 148 into reservoir 146. The excess fluid may fill reservoir 146. The fluid from reservoir 146 used with medical device 20. For example, the excess fluid may flow into umbilicus 7 of medical device 20 via tube 180. In such a way, the excess fluid may be used for delivering fluid to target tissue, for example, via medical device 20 or for washing a lens of an imaging device of medical device 20.

Although not shown, in some examples, second arm 148B may include a second gate. The second gate may be configured similar to that of first gate 176. The second gate may be comprised of a flexible material and may extend across an entirety of second lumen 152B of second arm 148B. The second gate may be configured to prevent or limit fluid flow into or out of second arm 148B and permit passage of lubricated medical instrument 120 through or past the second gate and into second arm 148B. For example, the second gate may be configured to prevent or limit a backflow of fluid from a medical device (e.g., medical device 20) fluidly coupled to second arm 148B. Additionally or alternatively, the second gate may be configured to prevent or limit the amount of fluid flowing into second arm 148B from fluid container 144. Thus, the second gate may be configured to permit passage of the lubricated medical instrument 120 through second arm 148B, while preventing or limiting additional or excess fluid flow into second arm 148B.

In some examples, port 143 (FIG. 2) may be configured to receive a medical instrument (e.g., medical instrument 120) such that medical device inserted 120 into port 143 is not wetted by wetting system 140. Medical instrument 120 may be inserted into port 143, through second branch 142B of conduit 142, and into third branch 142C of conduit 142. Medical instrument 120 may be inserted into opening 34 (FIG. 1) via third branch 142C.

One or more portions of wetting system 140 may be reusable and/or single-use. For example, fluid container 144 may be configured such that a user may fill or refill fluid container 144 one or more times. In some examples, valve 148 may be single-use or reusable. Additionally or alternatively, reservoir 146 may be single-use or multi-use. For example, reservoir 146 may be configured such that a user may remove the liquid captured by reservoir 146 and reuse reservoir 146. In such a way, components of wetting system 140 may be comprised of one or more materials capable of undergoing sterilization.

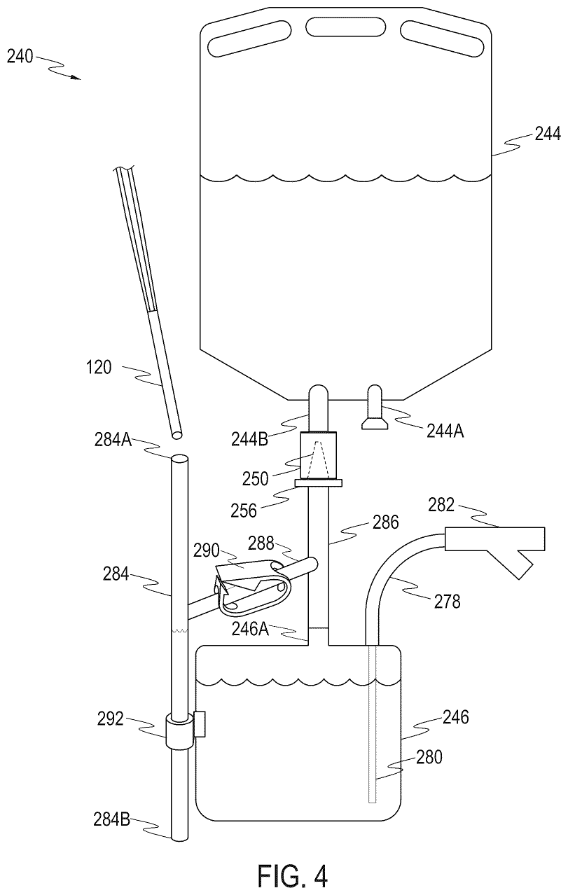

FIG. 4 illustrates a front view of an alternative wetting system 240. Wetting system 240 includes a fluid container 244, a reservoir 246, and a dipping container 284. Fluid container 244 may be in fluid communication with reservoir 246 via a lumen of a first conduit 286. First conduit 286 may extend between fluid container 244 and reservoir 246. First conduit 286 may permit fluid flow from fluid container 244 to reservoir 246.

Fluid container 244 may have any or all of the characteristics of fluid container 144, shown in FIG. 2. For example, fluid container 244 may include a first port 244A and a second port 244B. First port 244A may be configured to permit a user to inject fluid into or withdraw fluid from fluid container 244. Second port 244B may be configured to permit fluid flow to reservoir 246, for example, once second port 244B is punctured by a spike 250. Spike 250 may have any of the properties of spike 150 and may be configured to facilitate a fluid connection between fluid container 244 and first conduit 286. Spike 250 may be coupled to a proximal end of first conduit 286 or may be formed integrally with first fluid conduit 286. A flange 256 may have any of the properties of flange 156 and may be configured, for example, to prevent over-insertion of spike 250 into fluid container 244.

Reservoir 246 may be fluidly coupled to first conduit 286 by, for example, a port 246A of reservoir 246. In such a way, fluid from fluid container 244 may flow through first conduit 286 and into reservoir 246. Reservoir 246 may have any or all of the characteristics of reservoir 146, described above with respect to FIG. 2. For example, reservoir 246 may be any flexible or rigid container configured to contain a fluid.

In some examples, a first end of a second conduit 278 may be coupled to reservoir 246. A tube 280 may extend into reservoir 246 from second conduit 278. A second end of second conduit 278 may include an adapter 282 configured to couple to a pressure source (not shown) and/or to umbilicus 7 of medical device 20 (shown in FIG. 1). Second conduit 278, tube 280, and/or adapter 282 may have any or all of the characteristics of conduit 178, tube 180, and/or adapter 182, respectively, as described above with respect to FIG. 2. During a procedure, second conduit 278 may provide fluid from reservoir 246 to medical device 20 for, e.g., irrigation or lens washing.

A third conduit 288 may extend radially outward from a portion of first conduit 286. A lumen of third conduit 288 may be in fluid communication with a lumen of first conduit 286. Third conduit 288 may be configured to permit fluid flow between fluid container 244 to dipping container 284, for example, via first conduit 286 and third conduit 288. Third conduit 288 may be fixedly or removably coupled to dipping container 284.

Third conduit 288 may include a stopper 290 disposed around third conduit 288. Stopper 290 may be a clamp (e.g., a roller tube clamp), a valve, a clip, or any other mechanism used to control the flow of fluid through third conduit 288. For example, in a first, closed configuration, stopper 290 may be configured to prevent fluid flow to dipping container 284. In the closed configuration, stopper 290 may exert an inward force on one or more sides of third conduit 288. Stopper 290 may squeeze/compress third conduit 288, such that walls of third conduit 288 touch one another and close a lumen extending through third conduit 288. In other words, stopper 290 may pinch third conduit 288 closed. In a second, open configuration, stopper 290 may be configured to permit fluid flow through third conduit 288 and into dipping container 284. In the open configuration, stopper 290 may exert no inward force on a wall of third conduit 288 or may exert a force on third conduit 288 that is too small to close the lumen of third conduit 288. In some examples, stopper 290 may be configured for manual operation by a user. In other examples, stopper 290 may be electrically operated or operated via a control module (not pictured). In such examples, stopper 290 may configured to open and/or close using electrical inputs.

Dipping container 284 may be configured to hold or contain a fluid from fluid container 244. In some examples, dipping container 284 may be fixedly or removably coupled to reservoir 246 via coupler 292. For example, coupler 292 may be a clip or a clamp configured to permanently or temporarily couple dipping container 284 to reservoir 246. Coupler 292 may retain dipping container 284 in a desired (e.g., upright) configuration. In some examples, coupler 292 may include an adhesive or sticky material such that coupler 292 may temporarily or permanently adhere to reservoir 246.

Dipping container 284 may be shaped similar to a test tube. For example, dipping container 284 may have a first, open end 284A and a second, closed end 284B. A portion (or an entirety) of dipping container 284 may have a generally cylindrical shape.

Medical instrument 120 may be inserted into dipping container 284 via first, open end 284A. By inserting medical instrument into dipping container 284, medical instrument 120 may be wetted, or lubricated with fluid that has travelled into dipping container 284 from fluid container 244, via first conduit 286 and third conduit 288.

To use wetting system 240, a user may first spike second port 244B with spike 250. With stopper 290 on third conduit 288 in a closed position, fluid may begin to flow from fluid container 244 into reservoir 246 via first conduit 286. To fill dipping container 284, stopper 290 may be moved to an open position, thus permitting fluid flow through third conduit 288 and into dipping container 284. In such a way, dipping container 284 may be at least partially filled with fluid from fluid container 244. With stopper 290 in the open position, fluid flow into reservoir 246 from fluid container 244 may be reduced or stopped. Once dipping container 284 is filled with a desired amount of fluid (e.g., to a desired level of dipping container 284), stopper 290 may be moved to the closed position, thus stopping fluid flow into dipping container 284. With stopper 290 in the closed position, fluid from fluid container 244 may, once again, flow into reservoir 246 without any fluid being diverted to dipping container 284. Fluid contained within reservoir 246 may be used by medical device 20, for example, to supply fluids to target tissue and/or wash a lens of an imaging device. For example, as discussed above, second conduit 278 may be fluidly coupled to reservoir 246 and to one or more portions of medical device 20. Second conduit 278 may facilitate fluid flow from reservoir 246 and into medical device 20.

With dipping container 284 at least partially filled with fluid, medical instrument 120 may be inserted into dipping container 284 via first open end 284A. In such a way, medical instrument 120 may be wetted or lubricated. The lubricated medical instrument 120 may then be inserted into the patient and/or into a working channel of medical device 20 via, for example, opening 34 (shown in FIG. 1).

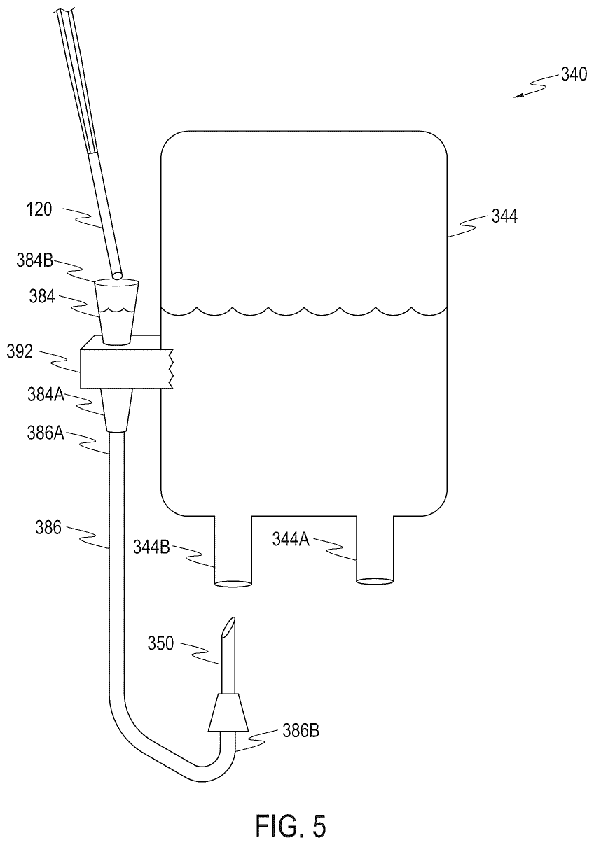

FIG. 5 illustrates an alternative wetting system 340. Wetting system 340 may include a fluid container 344 and a dipping container 384. Fluid container 344 may have any or all of the characteristics of fluid container 144 discussed with reference to FIG. 2 and/or fluid container 244 discussed with reference to FIG. 4. For example, fluid container 344 may have a first port 344A and a second port 344B. One or both of first port 344A and second port 344B may have any or all of the characteristics discussed above with respect to first port 144A and second port 144B of FIG. 2, and/or first port 244A and second port 244B of FIG. 4. Although a reservoir similar to reservoirs 146, 246 is not depicted in FIG. 5, it will be appreciated that wetting system 340 may also include a reservoir (having any properties of reservoirs 146, 246) coupled to fluid container 144 (e.g., via first port 344A).

Dipping container 384 may be fluidly coupled to fluid container 344 via a conduit 386. For example, a first end 386A of conduit 386 may be coupled to a first (bottom) end 384B of dipping container 384. Dipping container 384 may be similar to a small cup and may have any suitable shape (e.g., cone/frustum shaped, bowl shaped, cylindrical, etc.). As shown in FIG. 5, bottom end 384A of dipping container may have a diameter/width that is approximately the same as a diameter/width of conduit 386. Alternatively, bottom end 384A of dipping container 384 may be wider than conduit 386. Bottom end 384A of dipping container 384 may include an opening, such that fluid may flow through conduit 386, through the opening of bottom end 384A and into dipping container 384. In some examples, the opening of bottom end 384A may extend across an entire diameter/width of bottom end 384A. Alternatively, the opening of bottom end 384A may extend across only a portion of bottom end 384A. The opening of bottom end 384A may have a same width as a width of a lumen of conduit 386. Conduit 386 may be a separate element from dipping container 384 and affixed to bottom end 384A of dipping container 384. In alternatives, conduit 386 may be coupled to a side surface of dipping container 384, and a side wall of dipping container 384 may have an opening for fluidly coupling dipping container 384 to conduit 386.

Dipping container 384 may have a second (top) end 384B opposite bottom end 384A. Second end 384B may be at least partially open (e.g., open across a diameter/width of second end 384B). In some configurations, second end 384B may include a removable cap or top, for example, configured to open and close over the opening of second end 384B. For example, the user may open or remove the cap or top to insert medical instrument 120 into dipping container 384. Once the medical instrument is removed from dipping container 384, the user may then close or replace the cap or top to prevent spillage from dipping container 384. As shown in FIG. 5, walls of dipping container 384 may taper radially outward moving from bottom end 384A to second end 384B. However, such a shape is merely exemplary, and dipping container 384 may have any suitable shape.

A second end 386B of conduit 386 may include a needle 350 configured for insertion into second port 344B, thus permitting fluid flow between fluid container 344 and dipping container 384. In some examples, second port 344B may include a self-sealing silicone configured to be punctured multiple times by needle 350 without leaking. For example, second port 344B may be configured similar to a septum of a hypodermic port commonly used for chemotherapy treatments. In other configurations, needle 350 may be configured similar to and/or have any of the properties of spike 150 and/or spike 250. In such configurations, second port 344B may have any of the properties of second port 144B and/or second port 244B.

Once dipping container 384 is fluidly coupled to fluid container 344 via conduit 386, fluid may begin to flow into dipping container 384. Dipping container 384 may begin to fill according to the principle of communicating vessels. For example, dipping container 384 may begin to fill to a same level as the fluid in fluid container 344. In such a way, the fluid in dipping container 384 and fluid container 344 may both have a same fluid level.

Dipping container 384 may be removably coupled to fluid container 344 via a coupler 392. Coupler 392 may have any or all of the characteristics of coupler 292, discussed above with respect to FIG. 4. For example, coupler 392 may be a clip or a clamp configured to couple dipping container 384 to fluid container 346. In some examples, coupler 392 may be removably attached to fluid container 344 of FIG. 4 and/or dipping container 384. In other examples, coupler 392 may be fixedly coupled to dipping container 384 and removably coupled from fluid container 344. In further examples, coupler 392 may be fixedly coupled to fluid container 344 and removably coupled from dipping container 384. In some examples, coupler 392 may include an adhesive or sticky material such that coupler 392 may adhere to fluid container 344. For example, one or both of coupler 392 or fluid container 344 may include an adhesive material.

Dipping container 384 may be coupled to fluid container 344 anywhere on fluid container 344 (e.g., on a side of fluid container 344). For example, dipping container 384 may be positioned along a side of fluid container 344 according to a current fluid level of fluid container 344 and/or a fluid level desired in dipping container 384. For example, dipping container 384 may be positioned along a side of fluid container 344 such that second end 384B of dipping container 384 is above the level of the fluid contained within fluid container 344 and bottom end 384A is below the level of the fluid contained within fluid container 344. Thus, according to the principle of communicating vessels, fluid will flow into dipping container 384 but will not overflow out of second end 384B of fluid dipping container 384. If second end 384B were to be below the level of fluid contained within fluid container 344, dipping container 384 may overfill/overflow.

As the fluid level within fluid container 344 increases (e.g., by injecting fluid into fluid container 344 via first port 344A) or decreases, dipping container 384 may be repositioned on fluid container 344. Specifically, as the fluid level of fluid container 344 decreases, dipping container 384 may be moved downward on fluid container 344 such that dipping container 384 fills, as described above. As the fluid level of container increases, dipping container 384 may be moved upward relative to fluid container 344 to prevent spillage. Accordingly, a desired fluid level within dipping container 384 may be maintained.

Once dipping container 384 is filled to a desired level, medical instrument 120 may be inserted into dipping container 384. For example, a distal end of medical instrument 120 may be inserted into dipping container 384 such that the distal end of medical instrument 120 is wetted or lubricated. With medical instrument 120 wetted, medical instrument 120 may be inserted into the patient or medical device 20 via, for example, opening 34 (shown in FIG. 1).

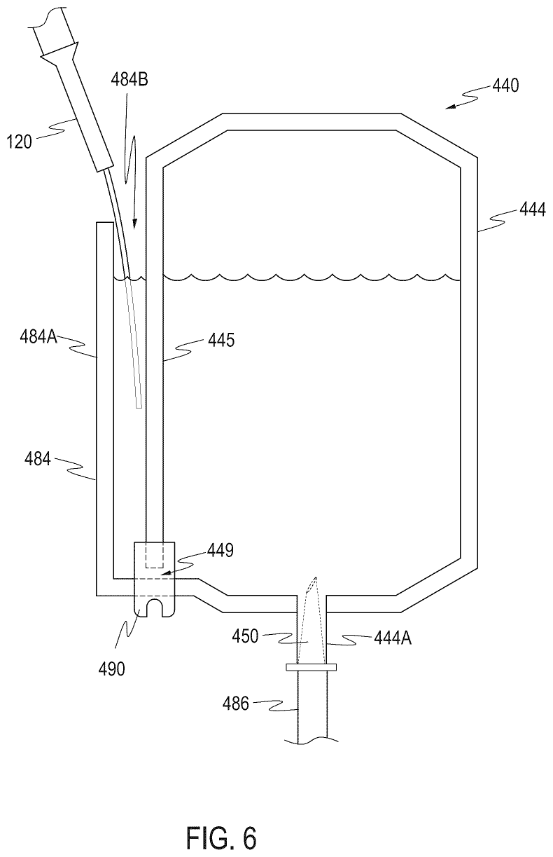

FIG. 6 illustrates an alternative wetting device 440. Wetting device 440 may include a fluid container 444 and a dipping portion 484. Fluid container 444 may have any or all of the characteristics described above with respect to fluid container 144 of FIG. 2, fluid container 244 of FIG. 4, and/or fluid container 344 of FIG. 5, except as described below. For example, fluid container 444 may be a bag or a bottle.

Dipping portion 484 may be integrally formed with fluid container 444. In some examples, dipping portion 484 may be formed from a same material as fluid container 444. In other examples, dipping portion 484 may be formed from a different material as fluid container 444. As shown in FIG. 6, an outer wall 484A of dipping portion 484 may extend along at least a portion of a side of fluid container 444. In some examples, outer wall 484A of dipping portion 484 may extend along an entire length of the side of fluid container 444, for example, between a top of fluid container 444 and a bottom of fluid container 444. Dipping portion 484 may have an opening 484B at a top end of dipping portion 484.

A wall 445 of fluid container 444 may separate dipping portion 484 from fluid container 444. For example, wall 445 may define one of the walls of each of fluid container 444 and dipping portion 484. Wall 445 may not extend along an entire length of fluid container 444 such that an opening 449 is formed to permit fluid communication between fluid container 444 and dipping portion 484. For example, as shown in FIG. 6, wall 445 may terminate above the bottom of fluid container 444/dipping portion 484, such that opening 449 is formed below wall 445. Alternatively, wall 445 may include an opening formed therein, above the bottom of wall 445. In some examples, wetting device 440 may be a bag, and wall 445 may be formed by fusing or coupling walls of the bag together. For example, wall 445 may be formed by using heat to fuse two sides wetting device 440. Alternatively, wall 445 may be formed using adhesives. In such a way, two sides of wetting device 440 may be fused or formed together to form fluid container 444 and dipping portion 484. Alternatively, fluid container 444, dipping portion 484, and wall 445 may be formed by any suitable method (e.g., molding, subtractive manufacturing, or additive manufacturing).

In some examples, fluid container 444 optionally includes a stopper 490. Stopper 490 may be disposed on or in fluid container 444 to control the flow of fluid between fluid container 444 and dipping portion 484. Stopper 490 may be a clamp, a valve, a clip, or any other mechanism used to control the flow of fluid from fluid container 444, into dipping portion 484. In some examples, stopper 490 may be a one-way valve, configured to permit fluid flow into dipping portion 484 and prevent fluid flow from dipping portion 484 into fluid container 444. In examples with stopper 490, outer wall 484A may extend along a portion of the side of fluid container 444 such that opening 484B of dipping portion 484 is below a top side of fluid container 444.

In a first, closed configuration, stopper 490 may be configured to prevent fluid flow from fluid container 444 into dipping portion 484. In a second, open configuration, stopper 490 may be configured to permit fluid flow from fluid container 444 into dipping portion 484. In some examples, stopper 490 may be configured for manual operation (opening/closing) by a user. In other examples, stopper 490 may be electrically operated or operated via a control module (not picture). In such a way, stopper 490 may configured to open and/or close using electrical inputs.

In some examples, a port 444A of fluid container 444 may be configured to be punctured by a spike 450 at an end of a conduit 486. Although not shown in FIG. 6, conduit 486 may be coupled to a reservoir having any of the properties of the reservoirs described above. Fluid from the reservoir may be utilized during a medical procedure, as described above. In alternatives, the reservoir may be omitted, and fluid from fluid container 444 may be transmitted to medical device 20 via conduit 486 for use during a procedure.

To fill dipping portion 484 with fluid, stopper 490 may be opened, thus permitting fluid to flow into dipping portion 484. Dipping portion 484 will fill according to the principle of communicating vessels. For example, dipping portion 484 may fill until a fluid level in dipping portion 484 is the same as a fluid level in fluid container 444. The amount of fluid in dipping portion 484 may be controlled, for example, via stopper 490. For example, if a user desires less fluid in dipping portion 484, stopper 490 may be closed, thus stopping fluid flow between fluid container 444 and dipping portion 484. In such a way, the amount of fluid in dipping portion 484 may be controlled. Alternatively, where stopper 490 is omitted, fluid may automatically flow into dipping portion 484 and may fill to a level of fluid in fluid container 444. In such alternatives, outer wall 484A of dipping portion 484 may extend along the side of fluid container 444 such that opening 484B of dipping portion 484 is above the fluid line in fluid container 444. In such a way, the fluid within dipping portion 484 may fill to a same level as the fluid in fluid container 444 without overflowing.

To lubricate medical instrument 120, medical instrument 120 may be inserted through opening 484B of dipping portion 484. For example, medical instrument 120 may be at least partially inserted into dipping portion 484 such that medical instrument 120 is wetted. Once medical instrument is wetted, medical instrument 120 may be inserted into a patient or through a working channel of medical device 20 (e.g., via opening 34), shown above with respect to FIG. 1.

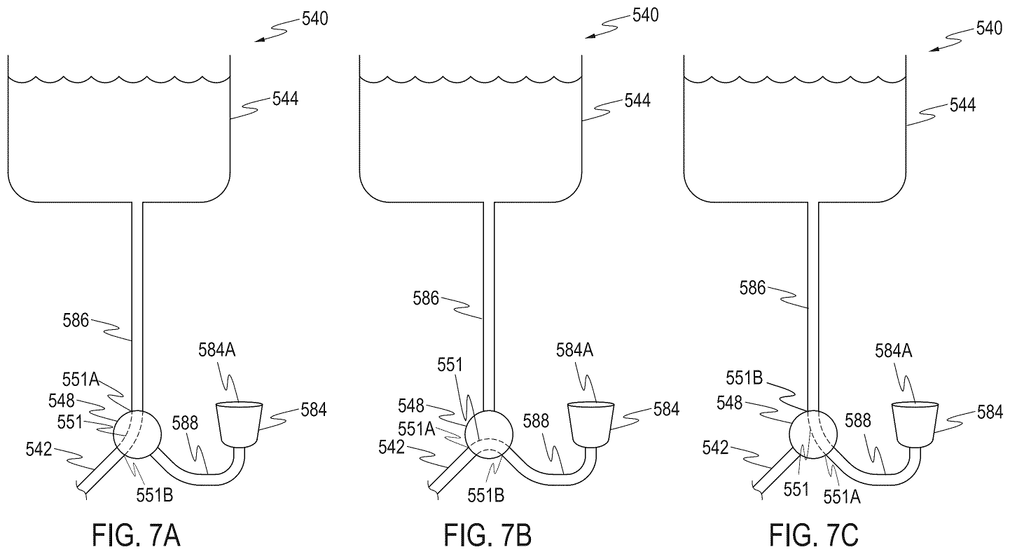

FIGS. 7A-7C illustrate an alternative wetting system 540 in a first configuration (FIG. 7A), a second configuration (FIG. 7B), and a third configuration (FIG. 7C). Wetting system 550 includes a fluid container 544, a movable valve 548, and a dipping container 584. Fluid container 544 may have any or all of the characteristics of fluid container 144 discussed with reference to FIG. 2, fluid container 244 discussed with reference to FIG. 4, fluid container 344 discussed with reference to FIG. 5, and/or fluid container 444 discussed with reference to FIG. 6. Dipping container may have any or all of the characteristics of dipping container 284 discussed with reference to FIG. 4 and/or dipping container 384 discussed with reference to FIG. 5.

In some configurations, dipping container 584 may include an opening 584A configured to receive at least a portion of a medical instrument (not shown). For example, during use, a portion of the medical instrument (not shown) may be inserted into dipping container 584 via opening 584A. Although not shown, in some configurations, dipping container 584 may be fixedly or removably coupled to fluid container 544 via, for example, a coupler. For example, the coupler may have any or all of the characteristics of coupler 292 discussed with reference to FIG. 4 and/or coupler 392 discussed with reference to FIG. 5.

A first conduit 586 may extend between fluid container 544 and valve 548. Although not depicted in FIGS. 7A-7C, first conduit 512 may have a spike or a needle (having any of the properties of the spikes or needle discussed above) or other structure for selectively joining first conduit 512 with fluid container 544 (e.g., with a port of fluid container 544).

A second conduit 542 may extend from valve 548 (e.g., radially outward from valve 548). Although not shown in FIGS. 7A-7C, second conduit 542 may be coupled to a reservoir having any of the properties of the reservoirs described above. Fluid from the reservoir may be utilized during a medical procedure, as described above. In alternatives, the reservoir may be omitted, and, second conduit 542 may extend to and be fluidly connected to a medical device, such as, for example, medical device 20 of FIG. 1.

A third conduit 588 may extend between and fluidly connect valve 548 and dipping container 584. As discussed above, with respect to dipping container 384, dipping container 584 may have an opening therein that is coupled to third conduit 588, such that third conduit 588 is in fluid communication with third conduit 588. For example, as described above, a bottom surface of dipping container 584 may include an opening that is joined to third conduit 588. In alternatives, dipping container 584 may be omitted, and third conduit 588 may terminate at a free end. A separate cup, bottle, or other type of container may be filled with fluid that flows through the free end of third conduit 588.

Valve 548 may include a lumen 551, which may define a path through which fluid may flow. As shown in FIGS. 7A-7C, valve 548 may have an approximately circular or spherical shape, and lumen 551 may have a curved path. Lumen 551 may extend from a first opening 551A on a surface of valve 548 to a second opening 551B, also on a surface of valve 548. In some examples, a central angle between first opening 551A and second opening 551B may be approximately 120 degrees.

In a first configuration, as shown in FIG. 7A, each end of lumen 551 may be aligned with first conduit 512 and second conduit 542. For example, first opening 551A may be aligned with first conduit 586, and second opening 551B may be aligned with second conduit 542. Thus, valve 548 may be configured to permit fluid flow from fluid container 544, through first conduit 586, and through second conduit 542 via lumen 551. In such a way, fluid from fluid container 544 may be delivered to a medical device (e.g., medical device 20). In the first configuration, because lumen 551 is misaligned with third conduit 588, fluid flow is not permitted to dipping container 584.

In a second configuration of valve 548, as shown in FIG. 7B, lumen 551 may be aligned with second conduit 542 and third conduit 588. For example, first opening 551A may be aligned with second conduit 542, and second opening 551B may be aligned with third conduit 588. In the second configuration, fluid may be permitted to flow between dipping container 584 and a reservoir or the medical device (e.g., medical device 20). Because dipping container may be above 584 and/or a pressure may not exist to move fluid upward from the reservoir or medical device into dipping container 584, in the second configuration, fluid may flow from dipping container 584, through third conduit 588, through lumen 551, and through second conduit 542. In such a way, dipping container 584 may be emptied. In the configuration of FIG. 7B, fluid flow from fluid container 544 may be turned off (not permitted).

In a third configuration of valve 548, as shown in FIG. 7C, lumen 551 may be aligned with first conduit 512 and third conduit 588. For example, first opening 551A may be aligned with third conduit 588, and second opening 551B may be aligned with first conduit 586. Thus, fluid may flow between fluid container 544 and dipping container 584. In situations in which a fluid level of dipping container 584 is lower than a fluid level of fluid container 544, fluid may flow from fluid container 544, into first conduit 586, through lumen 551, through third conduit 588, and into container 584, according to the principle of communicating vessels, as discussed above. In such a way, dipping container 584 may be filled with fluid from fluid container 544.

Valve 548 may be configured to transition, or move, between each configuration (e.g., first configuration, second configuration, and third configuration) by a variety of mechanisms. For example, rotation (e.g., clockwise or counterclockwise) of valve 548 may be transition valve 548 between each configuration. Additionally or alternatively, valve 548 may include one or more buttons, actuators, levers, etc., that are configured to transition valve 548 to each configuration. In some examples, valve 548 may be electrically operated. For example, valve 548 may be configured to transition between each configuration via electrical inputs. An arrangement and type of valve 548, described above, is merely exemplary, and any suitable type of valve may be utilized in order to fluidly couple the conduits, as described above. Valve 548 and/or conduits 542, 586, 588 may include one-way valves to prevent fluid from flowing in directions other than those described herein. In some examples, valve 548 may be a two-way valve, and the second configuration of FIG. 7B may be omitted.

To lubricate a medical instrument (not shown), dipping container 584 may be filled. For example, valve 548 may be moved to the third configuration (FIG. 7C) such that fluid from fluid container 544 flows into dipping container 584. Once dipping container 584 contains a desired volume of liquid, valve 548 may be moved to the first configuration (FIG. 7A). With fluid in dipping container 584, the medical instrument (not shown but having any of the features of medical instrument 120) may be inserted, or dipped, into dipping container 584 such that a portion of the medical instrument is wetted or lubricated. Once a desired length or portion of the medical instrument is wetted, the medical instrument may be inserted into the patient and/or inserted through a working channel of a medical device (e.g., medical device 20 of FIG. 1).

In order to fill a reservoir (not shown but having any of the features of any of the reservoirs shown and described above) or to allow fluid to flow to 44a medical device (e.g., medical device 20 of FIG. 1), valve 548 may be transitioned to or may remain in the first configuration.

In some instances, fluid from dipping container 584 may need or may be desired to be drained. In such an instance, valve 548 may be transitioned to the second configuration (FIG. 7B). In the second configuration, fluid is permitted to flow through valve 548 from dipping container 584 to, for example, a medical device and/or into a reservoir or disposal container.

Valve 548 may be moved between each configuration multiple times to achieve desired results. For example, valve 548 may be transitioned from the first configuration (FIG. 7A) to the third configuration (FIG. 7C) to fill and/or refill dipping container 584 one or more times throughout a procedure. Similarly, valve 548 may be transitioned to the second configuration (FIG. 7B) one or more times throughout the procedure, for example, to drain dipping container 584. Once dipping container 584 is drained, dipping container 584 may be replenished with fresh fluid by transitioning valve to the third configuration (FIG. 7C). In such a way, fresh fluid may periodically be provided to dipping container 584, such that the medical instrument (not shown) may be lubricated or wetted with fresh fluids.

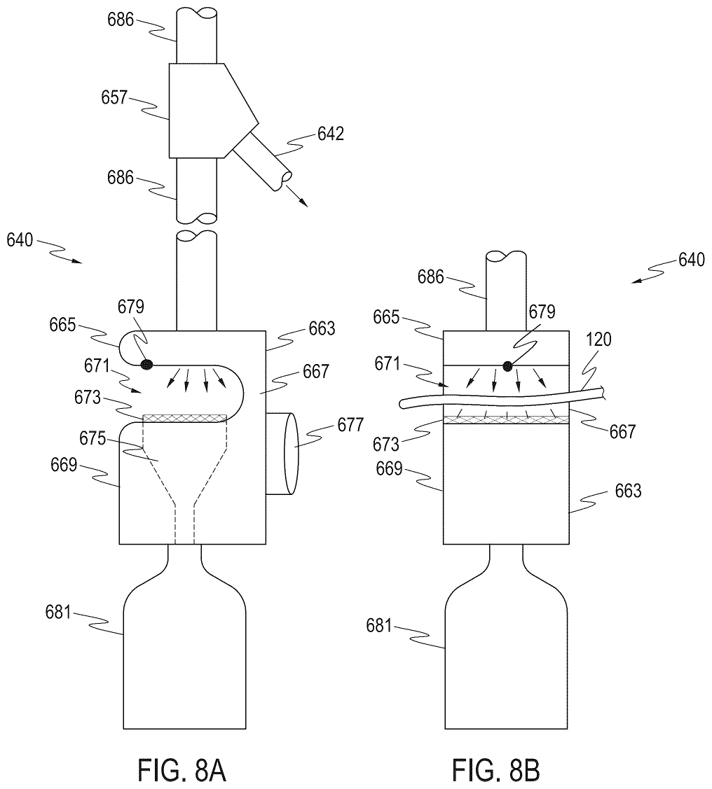

FIGS. 8A and 8B illustrate a front view (FIG. 8A) and a side view (FIG. 8B) of portions of an exemplary wetting system 640. Wetting system 640 may include a fluid reservoir 681 coupled to a wetting module 663. A conduit 686 may extend from an upper surface (or another surface) of wetting module 663. Conduit 686 may be fluidly coupled to a fluid source (not shown). Fluid source may be a fluid container such as, for example, any of the fluid containers previously discussed herein (e.g., fluid container 144, fluid container 244, fluid container 344, fluid container 444, and/or fluid container 544). In some examples, the fluid source may be a pressurized fluid source.

Conduit 686 may be coupled to an adapter 657. Adapter 657 may be configured permit fluid flow from the fluid source (a) through conduit 686, to wetting module 663 and (b) through a second conduit 642. Although not shown, second conduit 642 may be fluidly coupled to a medical device (e.g., medical device 20 of FIG. 1) or reservoir (having any of the features of any of the reservoirs disclosed herein). In some examples, adapter 657 may be configured to permit fluid flow through other conduits (not shown), for example, to accommodate fluid flow to multiple medical devices.

Wetting module 663 may include a nozzle arm 665 and a base 669. Nozzle arm 665 and base 669 may be coupled via a bridge portion 667. For example, nozzle arm 665 may extend laterally from bridge portion 667 in a first direction. Base 669 may extend laterally from bridge portion 667 in the same first direction. Nozzle arm 665 may be disposed above base 669 and spaced apart from base 669. In such a way, a gap 671 may be formed between nozzle arm 665 and base 669. Bridge portion 667 may form a first side of gap 671. In such a way, gap 671 may be C-shaped. For example, gap 671 may be defined by a bottom surface of nozzle arm 665, a top surface of base 669, and bridge portion 667. Gap 671 may be open on three sides, thus permitting a user to access gap 671 from at least three sides of wetting module 663 (e.g., a first side, a second side, and a third side). In such a way, gap 671 may be configured to receive a medical instrument 120 (shown in FIG. 8B).

In some examples, an absorbent material 673 may be disposed between nozzle arm 665 and base 669, for example, within gap 671. Absorbent material 673 may be a sponge-like material, for example, configured to retain moisture. In such a way, as fluid is delivered from nozzle arm 665, excess fluids may be retained by Absorbent material 673. In some examples, Absorbent material 673 may be on a lower side of gap 671 (for example, on an upper surface of base 669). In other examples, Absorbent material 673 may be on multiple sides of gap 671, for example, along a surface of bridge portion 667 and/or along a lower surface of nozzle arm 665.

Nozzle arm 665 may be configured to provide fluid through gap 671. For example, nozzle arm 665 may be configured to spray and/or drip fluid through gap 671. Excess fluid may be captured by a receptacle 675 disposed within base 669. In some examples, absorbent material 673 may cover receptacle 675. Receptacle 675 may be conical or funnel-shaped. For example, excess fluid from nozzle arm 665 may be captured by Receptacle 675. Receptacle 675 may be fluidly coupled to reservoir 681. Reservoir 681 may be fixedly or removably coupled to wetting module 663. In some examples, reservoir 681 may be removable such that, for example, reservoir 681 may be emptied and/or replaced. In other examples, reservoir 681 may be fixedly coupled to wetting module 663. In such an example, reservoir 681 may include a port or a drain such that reservoir 681 may be emptied or drained.

Wetting module 663 may include a controller 677 disposed on a rear surface of wetting module 663. Controller 677 may be configured to one or more aspects of the fluid flow. For example, controller 677 may control an amount of fluid being delivered (e.g., sprayed or dripped), the intensity of which the fluid is being delivered (e.g., a pressure), a temperature of the fluid, among other aspects. In some examples, controller 677 may be an on/off switch, configured to permit or stop the flow of fluid through nozzle arm 665. Controller 677 may include an actuator (e.g., a knob, a lever, a button, a switch, etc.). In some examples, controller 677 may be manually operated. For example, a user may manually adjust controller 677. In other examples, controller 677 may be operated via electrical inputs. For example, a computer (not shown) may be configured to adjust controller 677. In other examples, as discussed below, controller 677 may automatically activate a flow of nozzle arm 665 (e.g., based on sensing that medical instrument 120 is below nozzle arm 665).

As shown in FIG. 8B, during use, a portion of medical instrument 120 may be inserted through gap 671. As medical instrument 120 is moved through gap 671 and under nozzle arm 665, fluid may be delivered from nozzle arm 665 on to a surface of medical instrument 120. The fluid may be sprayed or dripped on to the surface of medical instrument 120. In such a way, the surface of medical instrument 120 may be wetted, or lubricated. Additionally or alternatively, medical instrument 120 may be moved along or through Absorbent material 673 such that Absorbent material 673 wets the surface of medical instrument 120.

Once the fluid is applied to a desired portion of medical instrument 120 such that medical instrument 120 is wetted or lubricated, medical instrument 120 may be removed from wetting module 663 and inserted into the patient or inserted through a working channel of a medical device (e.g., medical device 20 of FIG. 1).

In some examples, wetting module 663 may include one or more sensors 679. The one or more sensors 679 may be motion sensors, for example, configured to recognize when medical instrument 120 is passed through gap 671. The one or more sensors 679 may be electrically coupled to controller 677. For example, as medical instrument 120 is passed through gap 671, sensor(s) 679 may provide an electrical signal to controller 677 to turn nozzle arm 665 on to deliver fluid. Once medical instrument 120 is removed from gap 671, sensor(s) 679 may be configured to provide an electrical signal to controller 677 to turn off nozzle arm 665 such that fluid is no longer being delivered.

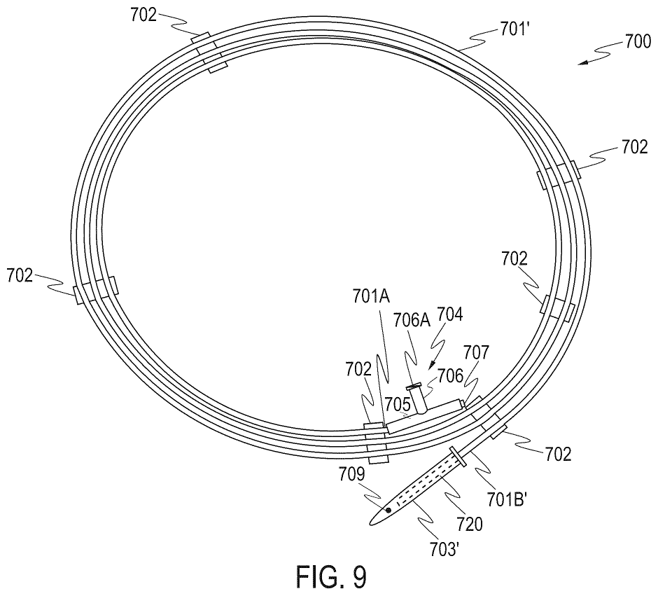

FIG. 9 illustrates a hoop 700. Hoop 700 includes a looped tube 701 held in place by a plurality of clips 702. A first end 701A of tube 701 may include an adapter 704. Adapter 704 may define one or more lumens that may be in fluid communication with a lumen of tube 701. Adapter 704 may be configured to be removably or fixedly coupled to a fluid source (not shown). For example, adapter 704 may include a shaft 705 and an arm 706. Arm 706 may extend approximately perpendicularly from shaft 705. Alternatively, arm 706 may extend at any suitable angle from shaft 705. Adapter 704, including shaft 705 and arm 706, may have a “T” shape. Arm 706 and shaft 705 each may have a lumen extending therethrough. The lumens of arm 706 and shaft 705 may be in fluid communication with one another. Arm 706 may include an opening 706A on a free end of arm 706 (an end of arm that is not coupled to shaft 705). Opening 706A may provide access to the lumen of arm 706.

Opening 706A may be selectively coupled to a source of fluid (not shown). In some examples, the fluid source may be a syringe of fluid. The syringe may be coupled to arm 706 of adapter 704, such that water may be injected into the lumens of adapter 704 and, thus, tube 701. In some examples, adapter 704 may include a seal 707 on at least one end of adapter 704. Seal 707 may be configured to prevent fluid from flowing out of tube 701. In some configurations, seal 707 may include an opening through which at least a portion of medical instrument may extend. For example, seal 707 be between arm 706 and an open side of shaft 705. Seal 707 may extend around, or at least partially surround medical instrument 720. In such a way, seal 707 may form a liquid-tight seal around medical instrument 720.

701 may include a cap 703 fixedly or removably coupled to a second end 701B of tube 701. Cap 703 may be configured to prevent fluids from escaping tube 701. In such a way, medical instrument 720 may be wetted, or lubricated, prior to being removed from tube 701. Cap 703 may include a pressure release valve 709. For example, as liquid is injected into tube 701, pressure release valve 709 may release air trapped within tube 701. Once a desired volume of liquid is injected into tube 701, cap 703 may be removed and a wetted or lubricated medical instrument 720 may be removed from tube 701.

It will be apparent to those skilled in the art that various modifications and variations can be made to the disclosed device without departing from the scope of the disclosure. In some examples, various components discussed herein may be used interchangeably with each embodiment. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A medical system, comprising:
a fluid source;
a fluid reservoir; and
a valve coupled to the fluid source and the fluid reservoir so as to permit fluid flow between the fluid source and the fluid reservoir,
wherein the valve includes a first opening configured to receive a medical instrument, and
wherein, upon insertion of the medical instrument into the first opening of the valve, the medical instrument is lubricated with a fluid from the fluid source.

2. The medical system of claim 1, wherein the medical system further includes an adapter, wherein a first end of the adapter is coupled to the first opening of the valve, wherein a second end of the adapter includes an opening configured to receive the medical instrument, and wherein the adapter includes a gate configured to prevent fluid flow from the first end of the adapter to the second end of the adapter.

3. The medical system of claim 2, wherein the gate includes a first end and a second end, wherein the first end is fixed to a wall of the adapter, wherein the second end of the gate is free.

4. The medical system of claim 3, wherein, in a first configuration, the second end of the gate abuts a wall of a lumen of the adapter and, upon insertion of the medical instrument into the adapter and through the gate, the gate is transitioned to a second configuration,
wherein, in the second configuration, the second end of the gate abuts a surface of the medical instrument, and wherein, in the second configuration, the gate is configured to prevent fluid flow from the first end of adapter to the second end of the adapter.

5. The medical system of claim 3, wherein, the second end of the gate is between the first end of the adapter and the first end of the gate.

6. The medical system of claim 2, wherein the adapter includes a channel extending through a wall of the adapter.

7. The medical system of claim 6, wherein the channel extends along at least a portion of a length of the adapter.

8. The medical system of claim 2, wherein the adapter is integrally formed with the valve.

9. The medical system of claim 1, wherein the valve further includes:
a first arm coupled to the fluid source;
a second arm coupled to the fluid reservoir;
a third arm, wherein the third arm includes the first opening configured to receive the medical instrument; and
a fourth arm, wherein the fourth arm includes a second opening,
wherein each of the first arm, the second arm, the third arm, and the fourth arm are fluidly connected.

10. The medical system of claim 9, wherein a longitudinal axis of the third arm is angled in a first direction relative to a longitudinal axis of the first arm, wherein a longitudinal axis of the fourth arm is angled in a second direction, relative to the longitudinal axis of the first arm.

11. The medical system of claim 10, wherein the longitudinal axis of the first arm and a longitudinal axis of the fourth arm are parallel or coaxial, and
wherein the longitudinal axis of the second arm and the longitudinal axis of the third arm are parallel or coaxial.

12. The medical system of claim 10, wherein the first direction of the third arm is opposite the second direction of the fourth arm.

13. The medical system of claim 10, wherein, the fluid reservoir is fluidly coupled to a medical device.

14. The medical system of claim 9, wherein a conduit is coupled to the second opening of the fourth arm.

15. The medical system of claim 14, wherein the conduit includes a first branch, a second branch, and a third branch, wherein the first branch is coupled to the fourth arm, wherein the second branch includes an opening configured to receive a second medical instrument, and wherein the third branch is coupled to an opening of a medical device having a working channel.

16. A medical system, the medical system comprising:
a fluid container; and
a dipping container configured to receive at least a portion of a medical instrument, wherein the dipping container includes a first opening in a bottom or a side of the dipping container, and wherein the dipping container includes a second opening configured for receiving a medical instrument, and
wherein the fluid container is fluidly coupled to the dipping container via the first opening.

17. The medical system of claim 16, wherein the dipping container is movable relative to the fluid container.

18. The medical system of claim 16, further comprising a stopper, wherein, the stopper is configured to prevent fluid flow to the dipping container in a first configuration, and wherein, the stopper is configured to permit fluid flow to the dipping container in a second configuration.

19. A medical device, the medical device comprising:
a bridge;
a base extend laterally from the bridge in a first direction;
an arm extending laterally from the bridge in the first direction; and
a gap between the base and the arm,
wherein the arm includes a nozzle configured to deliver a fluid into the gap, wherein, upon insertion of a medical instrument through the gap, the fluid is applied to a surface of the medical instrument.

20. The medical device of claim 19, wherein the medical device includes a controller, wherein the controller is configured to control delivery of the fluid.
